(19) United States
(12) Patent Application Publication
(10) Pub. No.: US 2016/0088853 A1
(43) Pub. Date: Mar. 31, 2016

(54) Ultraviolet Disinfection of Produce, Liquids and Surfaces

(71) Applicants: DREXEL UNIVERSITY, Philadelphia, PA (US); THE REGENTS OF THE UNIVERSITY OF CALIFORNIA, Oakland, CA (US)

(72) Inventors: Rohan Vijay Tikekar, Bryn Mawr, PA (US); Nitin Nitin, Davis, CA (US); Aachen Elsinghorst, Cherry Hill, NJ (US)

(73) Assignees: THE REGENTS OF THE UNIVERSITY OF CALIFORNIA, Oakland, CA (US); DREXEL UNIVERSITY, Philadelphia, PA (US)

(21) Appl. No.: 14/784,407

(22) PCT Filed: Apr. 16, 2014
(86) PCT No.: PCT/US14/34291

§ 371 Date: Oct. 14, 2015

Related U.S. Application Data
(60) Provisional application No. 61/813,160, filed on Apr. 17, 2013.

(51) Int. Cl. A23B 7/015; A61L 2/08; C02F 1/72; A23B 7/10; C02F 1/32

(52) U.S. Cl. CPC A23B 7/015; A23B 7/10; C02F 1/32; C02F 1/725; A61L 2/088; C02F 2303/04; C02F 2305/023; C02F 2305/10; A23V 2002/00

(57) ABSTRACT

The present invention is directed to a process of disinfecting produce, comprising the steps of associating the produce with one or more photosensitizers selected from the group consisting of gallic acid, fructose, riboflavin, sodium chlorophyllin and photo-porphyrin; and exposing the associated produce and one or more photosensitizers to UV radiation sufficient to cause the one or more photosensitizers to generate one or more free radicals. The produce may be fresh produce and may be selected from fresh vegetable and fruits. The present invention may also be used to treat waste water by adding at least one photosensitizer to waste water ant then expose the waste water to US radiation.

### RELATED APPLICATION DATA

This application claims priority to International Application No. PCT/US14/34291, filed Apr. 16, 2014, and U.S. Provisional Application No. 61/813,160, filed Apr. 17, 2013, the contents of which are hereby incorporated by reference.

### 1. FIELD OF THE INVENTION

The present invention relates generally to disinfection of produce, liquids and surfaces. In particular, the present invention is directed to a method of disinfecting produce, liquids and surfaces by associating the produce, liquids ad surfaces with one or more photosensitizers and exposing the one or more photosensitizers to ultraviolet light.

### 2. DESCRIPTION OF THE RELATED TECHNOLOGY

Fresh produce has been associated with frequent microbial outbreaks. This is a global problem, including in the industrialized countries. For example, in the United States of America, according to the U.S. Centers for Disease Control, 46% of all food-borne illnesses between the years 1998 and 2008 were due to contamination of fresh produce, especially green leafy vegetables. In 2012 alone, 3 major multi-state microbial outbreaks were associated with contamination of fresh produce such as spinach, cantaloupes and mangoes. The causes of outbreaks linked to fresh produce contamination have been attributed to poor worker hygiene, cross-contamination and improper sanitation of fresh produce.

Fresh produce is routinely sanitized by a washing operation. The wash water used usually contains hypochlorite salt as a sanitizer. Other sanitizers used include hydrogen peroxide and oxidizing chemicals such as electrolyzed water, ozone and antimicrobial oils. However, the use of wash water containing sanitizers such as sodium hypochlorite typically reduces the microbial load only by 1 to 2 log values and is usually ineffective in inactivating microorganisms internalized within the fresh product. The activity of these sanitizers is seriously affected by various factors such as the pH of the water used in the wash and the presence of an organic load in the wash water. Acidified sodium chlorite is capable of achieving up to a 5 log reduction of E. coli 0157:H7 in lettuce but this sanitizer cannot be used for products such as cut vegetables and fruits that are ready for consumption due to the undesirable residual taste associated therewith.

Furthermore, these chemical based sanitizers have the drawback of potentially leaving toxic substances on the fresh produce. There is a general trend towards lowering or eliminating chlorine from washing operations due to health and environmental concerns. The majority of these sanitizers are also relatively ineffective for reducing viral counts. In addition, these chemicals may form degradation products which may be unacceptable to some persons or irritating to persons who may have allergies or are otherwise sensitive to such materials.

Ultraviolet (UV) light based technologies have the potential to inactivate pathogens in food systems while not leaving any residual substances on the food, and, at the same time, maintaining the attributes of the food that provide nutrition and ensure quality. These technologies have been used to reduce the microbial load in food systems, particularly in beverage products and fresh produce. But the application of UV technologies has been limited because of, for example, the low penetration depth of UV light into the food matrix, which is typically about 0.1 to 1 cm from the surface of the depending upon the nature of the food matrix. This limited penetration depth is due to interactions between the UV light and UV absorbing compounds in the food matrix, as well as the UV light being scattered by components of the food matrix. The ineffectiveness of disinfecting produce by UV radiation may also be due to the rough and contoured shapes of some solid foods, which allow microorganisms to survive within the crevices and shadows that prevent a homogenous UV light treatment of the product.

U.S. Patent Application Publication No. 2003/0035750 discloses a process of using photosensitizers exposed to an illumination energy for providing antibacterial treatment of surfaces on consumer and industrial items. The illumination energy and its intensity levels are sufficient to transform the photosensitizers to singlet oxygen which destroys at least a substantial proportion of the targeted microbes on the surfaces. It is also possible to select photosensitizers that are activated only by certain wavelengths prominently present in some forms of illumination, such as those lamps commonly present in a laboratories, medical offices, pharmacies and food service areas, thereby enabling antimicrobial treatment of the surfaces on demand when the lamps are turned on.

U.S. Patent Application Publication No. 2003/0215784 discloses a method for inactivation of microorganisms in fluids or on surfaces. The method includes the steps of applying an effective, non-toxic amount of an endogenous photosensitizer to the surface and exposing the surface and photosensitizer to photoradiation sufficient to transform the endogenous photosensitizer to free radicals that inactivate at least some of the microorganisms. The surfaces that may be treated by this method include surfaces of foods, animal carcasses, wounds, food preparation surfaces and bathing and washing vessel surfaces. Alloxazines and K- and L-vitamins are among the preferred endogenous photosensitizers. Systems and apparatuses for flow-through and batch processes are also provided for decontamination of the objects.

U.S. Patent Application Publication No. 2004/0219057 discloses a method of deactivating biological agents on a surface. The method includes aerosol spraying of the surface with an electrostatically charged solution, and then illuminating the surface with UV light. The solution contains a sufficient amount of a photosensitizer for deactivating at least some biological agents.

Matins et al., “Antimicrobial efficacy of riboflavin/UVA combination (365 nm) in vitro for bacterial and fungal isolates: a potential new treatment for infectious keratitis,” Investigative Ophthalmology & Visual Science, vol. 49, pages 3402-3408, 2008, discloses a process for inactivating bacteria and fungi on an artificial surface. Riboflavin is used as a photosensitizer and is spread on the artificial surface, which is then exposed to UV light of a wavelength of 365 nm. The method is effective in inactivating most of the bacterial and fungal strains on the artificial surface.

Tikekar et al. (“Patulin Degradation in a Model Apple Juice System and in Apple Juice during Ultraviolet Processing,” Journal of Food Processing and Preservation, DOI: 10.1111/jfpp.12047, Dec. 7, 2012) shows that in the presence of fructose, the UV induced rate of degradation of patulin (a mycotoxin) increased in a model apple juice system. This effect may be attributed to oxidative stress from free radicals produced by fructose upon the exposure to UV radiation. The UV induced free radicals generated by fructose seem to be oxidative in nature, thus capable of oxidizing food components.

Another study (Triantaphylides et al. “Photolysis of D-fructose in aqueous solution,” Carbohyd. Res., vol. 100, pp. 131-141, 1982) showed that the photolysis of fructose can lead to formation of hydroxyalkyl and acyl radicals, which after a reaction with atmospheric oxygen leads to a formation of peroxyl and superoxide radicals. These reactive oxygen species are known to generate oxidative stress within cells and lead to death (Martinez et al., “Fluoroquinolone Antimicrobials: Singlet Oxygen, Superoxide and Phototoxicity,” Photochem. Photobiol., vol. 67, p. 399, 1998; Lian et al., “Blue light induced free radicals from riboflavin on E. coli DNA damage,” J. Photochem. Photobiol. B: Biology, vol. 119, pp. 60-64, 2013; Sies H., “Physiological society symposium: impaired endothelial and smooth muscle cell function in oxidative stress,” Experimental Physiology, vol. 82, pp. 291-295 (1997).

### SUMMARY OF THE INVENTION

The present invention provides an improved process for treatment of a surface of a produce or a medical device by using UV light in combination with one or more photosensitizers to inactivate at least some microbes such as bacteria and viruses in wash water and/or on the produce or medical device.

In one aspect, the present invention is directed to a process for the treatment of a surface selected from a surface of produce and surface of a medical device, comprising the steps of associating the surface with one or more photosensitizers selected from the group consisting of gallic acid, riboflavin, photo-porphyrin, sodium chlorophyllin, fructose; and exposing the associated one or more photosensitizers and the surface to ultraviolet radiation to cause the at least one photosensitizer to generate one or more free radicals.

A process for treatment of waste water, comprising the steps of adding at least one photosensitizer selected from the group consisting of selected from the group consisting of gallic acid, riboflavin, photo-porphyrin, sodium chlorophyllin and fructose; and exposing the waste water with the at least one photosensitizer to UV radiation to cause the at least one photosensitizer to generate one or more free radicals.

### BRIEF DESCRIPTION OF THE DRAWINGS

FIG. 1 depicts a schematic view of a process of inactivating microorganisms according to one embodiment of the present invention.

FIG. 2 shows the effect of UV light on the fluorescence intensity of fluorescein dye in the present or absence (control) of 0.4 w/v % fructose.

FIG. 3 shows the effect of storage on the fluorescence intensity of fluorescein solution containing 0.4 w/v % fructose after UV light treatment.

FIG. 4 shows the effect of ascorbic acid on UV light induced fluorescence loss in a 0.4 w/v % fructose solution.

FIG. 5 shows the effect of fructose concentration on the UV light induced inactivation rate of ascorbic acid.

FIG. 6 shows that using gallic acid as photosensitizer with exposure to UV light treatment is more effective in reducing microbial counts than using UV light alone.

FIG. 7 shows wide-field bioluminescence imaging used to characterized removal of bacterial cells from fresh lettuce leaf disks using a simple washing procedure.

FIG. 8 shows the correlation between bioluminescence intensity and plate count in a lettuce sample with E. coli.

FIG. 9 shows inactivation of MS2 viral particles using UV radiation.

FIGS. 10A-10B show relative decay of fluorescein as a function of duration of exposure to UV light in aqueous solutions containing no sugar (control), sucrose (263 mM), glucose (500 mM) or fructose (500 mM) and (10A) in presence of various concentrations (10-500 mM) of fructose (10B). Each data point is an average of triplicate measurements±standard deviation.

FIG. 11 shows relative fluorescence decay of fluorescein as a function of duration of exposure to UV light in 1 μM fluorescein solutions containing 33, 66 and 132 μM furan or 10 mM fructose. Each data point is an average of triplicate measurements±standard deviation.

FIG. 12 shows relative fluorescence decay of fluorescein as a function of duration of exposure to UV light in a 20 mM fructose solution containing 0, 25 and 50 μM of ascorbic acid. Each data point is an average of triplicate measurements±standard deviation.

FIG. 13 shows relative fluorescence decay of fluorescein upon exposure to UV light for 60 seconds in 500 mM fructose aqueous solutions either purged or not purged with nitrogen. Each data point is an average of triplicate measurements±standard deviation.

FIG. 14 shows relative fluorescence decay of fluorescein as a function of duration of exposure to UV light in an aqueous solution of 100 mM fructose or of 294 μM hydrogen peroxide. Each data point is an average of triplicate measurements±standard deviation.

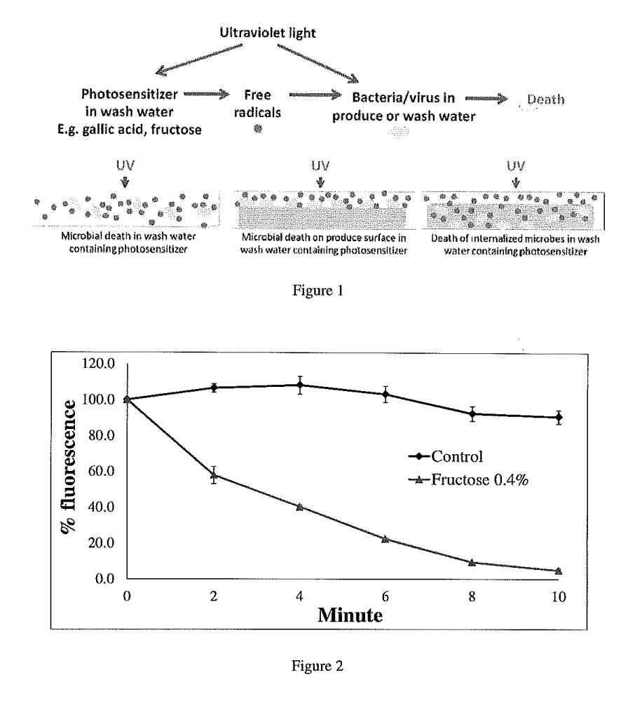

### DETAILED DESCRIPTION OF THE INVENTION

For illustrative purposes, the principles of the present invention are described by referencing various exemplary embodiments. Although certain embodiments of the invention are specifically described herein, one of ordinary skill in the art will readily recognize that the same principles are equally applicable to, and can be employed in other systems and methods. Before explaining the disclosed embodiments of the present invention in detail, it is to be understood that the invention is not limited in its application to the details of any particular embodiment shown. Additionally, the terminology used herein is for the purpose of description and not of limitation. Furthermore, although certain methods are described with reference to steps that are presented herein in a certain order, in many instances, these steps may be performed in any order as may be appreciated by one skilled in the art; the novel method is therefore not limited to the particular arrangement of steps disclosed herein.

It must be noted that as used herein and in the appended claims, the singular forms “a”, “an”, and “the” include plural references unless the context clearly dictates otherwise. Furthermore, the terms “a” (or “an”), “one or more” and “at least one” can be used interchangeably herein. The terms “comprising”, “including”, “having” and “constructed from” can also be used interchangeably.

As use herein, the term, “produce” refers to agricultural products which are generally in the same state as when they were harvested. Produce includes fresh produce such as fresh fruits, cut fruits and vegetables.

The term, “fresh” indicates that a product has not been cooked, dried, or frozen.

In one aspect, the present invention relates to a process for treatment of a surface selected from a surface of produce and a surface of a medical device, comprising the steps of associating the surface with one or more photosensitizers and exposing the one or more photosensitizers to ultraviolet (UV) radiation. The UV radiation induces the one or more photosensitizers to produce one or more free radicals, which in turn inactivate microorganisms that may be present. The microorganisms that may be inactivated by the method of the present invention include bacteria, fungi, and viruses.

Associating the surface with one or more photosensitizers may be accomplished in a variety of ways. For example, the surface may be contacted with the photosensitizer or with a composition comprising the photosensitizer. The composition comprising the photosensitizer, also referred to herein as a wash composition, may be a solution, a dispersion or a suspension of the photosensitizer in a fluid. It is important that the photosensitizer is located in sufficiently close proximity to the surface, so that free radicals generated by irradiation of the photosensitizer with UV radiation can propagate to the surface and come into contact with microbes located on the surface or even under the surface.

In some embodiments, the surface to be treated is a surface of produce. One suitable method for associating or contacting the photosensitizer with the surface of produce involves partial or complete immersion of the produce in a wash composition containing the photosensitizer. The immersion of the produce may be accomplished in a tank. Alternative suitable methods of contacting the photosensitizer with the surface of the produce include spraying the wash composition onto the produce, dipping the produce in the wash composition, wiping the wash composition onto the produce, or other means known to a skilled person. In some embodiments, the produce is covered entirely by the wash composition in order to ensure exposure of the entire surface of the produce. The free radicals generated by UV light exposure will thus come into contact with the surface of the produce, and in some cases will penetrate under the surface of some produce.

An electrically-powered pumped sprayer, an electro-sprayer, or a simple manually pumped sprayer may be used to spray wash composition onto the produce. In some embodiments, a fogger or canister for delivery of the spray may also be used.

In some embodiments, the surface to be treated is a surface of a medical device. Some small medical devices may be partially or completely immersed in a wash composition containing the one or more photosensitizers. For some large medical devices or medical devices that are not suitable for immersion in a wash composition, the photosensitizer may be sprayed onto the surface of the medical devices. Disinfection or sterilization of medical devices by associating the surface of the medical device with the photosensitizers followed by exposure to UV light is especially important for those devices which cannot be autoclaved, or otherwise sterilized by presently known means.

Photosensitizers suitable for use in the present invention include gallic acid, riboflavin, photo-porphyrin, sodium chlorophyllin and fructose. Gallic acid, also known as 3,4,5-trihydroxybenzoic acid, has the formula:

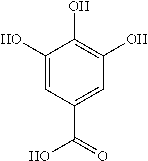 [SMILES: O=C(O)c1cc(O)c(O)c(O)c1]

Fructose is a 6-carbon polyhydroxyketone, which is an isomer of glucose and has the molecular formula C₆H₁₂O₆. Both D-fructose and L-fructose may be used in the present invention. In some embodiments, D-fructose is used.

The photosensitizers, when exposed to UV radiation, undergo photolysis and form one or more free radicals which inactivate microorganisms. For example, fructose may form hydroxyalkyl radicals, acyl radicals, and peroxyl radicals as a result of exposure to UV radiation. Gallic acid may form hydroxyl radicals when exposed to UV radiation.

One advantage of the photosensitizers of the present invention is that they only generate a significant amount of free radicals that induce oxidative stress upon exposure to UV radiation. This allows for a better control of the process than is the case with some prior art materials which may generate a significant amount of free radicals even in the absence of UV radiation.

Another advantage of the use of the photosensitizers of the present invention is that they can be formulated in a solution that remains relatively stable over time, which allows for storage and shipping of solutions of the photosensitizers, facilitating distribution, handling and use thereof.

The free radicals produced by the photosensitizers of the present invention are capable of inactivating microorganisms, such as by interfering with pathways in the microorganisms to prevent their replication. In particular, some free radicals may bind to one or more nucleic acids in the microorganisms. “Nucleic acid” includes ribonucleic acid (RNA) and deoxyribonucleic acid (DNA). Other free radicals may act by binding to cell membranes or by other mechanisms, thus destroying the microorganism structure. The present invention is not limited to use of a particular mechanism for microorganism inactivation but rather may include one or more different mechanisms which may be the result the various types of free radicals generated by the photosensitizes of the invention.

The microorganisms that can be inactivated by free radicals include bacteria, viruses and fungi. The microorganisms may be on the surface of the produce or internalized in the produce. Because the free radicals generated by the photosensitizers of the present invention are capable of penetrating into certain types of produce, the internalized microorganisms can be more effectively inactivated by the present method than by use of UV radiation alone.

In some embodiments, the free radicals generated by the photosensitizers of the invention may also be effective for destroying pesticides by oxidizing them. This may provide additional advantages because some produce may have pesticides on the surface. The method of the present invention may thus oxidize the pesticides to make them no longer harmful to humans.

Examples of the wash compositions in which the photosensitizer may be delivered include a solution, a suspension, and an emulsion. In one embodiment, the solution is an aqueous solution. Any suspension comprising the particles of the insoluble photosensitizer is appropriate for use in the invention, provided that the suspension is stable under the conditions that it is stored and used. The wash composition may comprise micelles which comprise the photosensitizer to deliver the photosensitizer in a controlled released manner. Emulsions may be used for less water soluble photosensitizers. Other suitable solvents may also be used. Some other examples of solvents include alcohols, glycerol, dimethyl sulfoxide and other polar solvents. Preferably, solvents approved or safe for use in food treatment are employed. The solvent or carrier for the photosensitizer should be chosen to avoid blocking or consuming the free radicals. The use of materials oxidizable by the free radicals generated by the photosensitizer should be avoided.

Also, the use of materials that may otherwise interfere with propagation of generated free radicals to the microbes should avoided. For example, it may be desirable to minimize the organic load in a wash composition containing the one or more photosensitizers. For example, it may be desirable to maintain the organic load in a wash composition below 1000 ppm, or below 600 ppm or below 400 ppm.

The wash composition preferably has a pH at which the photosensitizers are relatively stable. A skilled person will appreciate that the pH may need to be adjusted according to the photosensitizer employed in the solution by using an appropriate buffer solution, because different photosensitizers may have different pH ranges in which they are stable.

In addition, the efficiency of photolysis of the photosensitizer when exposed to UV radiation may also be considered when determining a suitable pH for the wash composition. In general, the pH of the wash composition will typically be in a range of from about 3 to about 7 or from about 4 to about 7. For example, the pH for a suitable aqueous solution of gallic acid, fructose, sodium chlorophyllin, riboflavin or photo-porphyrin may be in the range of from 3 to 7 or from 4 to 7.

The photosensitizer concentration in the wash composition may be in the range of from about 0.1 w/v % to about 10 w/v %, or from about 0.2 w/v % to about 5 w/v %, or from about 0.5 w/v % to about 3 w/v %. If the photosensitizer generates a larger amount of free radicals per unit weight of the photosensitizer when exposed to UV radiation, a lower concentration of photosensitizer may be used. On the other hand, if the photosensitizer generates smaller amount of free radicals per unit weight of the photosensitizer when exposed to UV radiation, use of a higher concentration of photosensitizer in the solution may be appropriate.

The photosensitizer concentration in the wash composition may also vary according to the means by which the surface is to be associated with the wash composition. For example, if the produce or medical device is immersed in the wash composition, a lower photosensitizer concentration may be used than, for example, in the case of spraying the produce or medical device with a wash composition.

The concentration of the photosensitizer employed may also be varied based on the type of produce or medical device being disinfected. Free radicals are capable of oxidizing food components and thus may affect the quality of certain types of produce due to oxidative damage. Different types of produce may have different levels of tolerance to oxidative stress. A skilled person can readily determine a suitable photosensitizer concentration to be used for a particular type of produce by assessing the level of oxidative damage caused by the process.

The temperature employed during association of the produce with photosensitizer may be sufficiently low such that the produce does not substantially change in appearance, nutritional content, or taste upon exposure to that temperature. Suitable temperatures for the association step may be in the range of from about 0° C. to about 10° C. for produce that is refrigerated. Suitable temperatures for the association step may be in the range of from about 10° C. to about 50° C., or from about 15° C. to about 30° C. for fresh produce. The temperature for treatment of the surface of medical devices may be at ambient temperature.

The wash composition may be prepared immediately before the association step. This method can potentially be used to minimize loss of photosensitizers to degradation during storage. The photosensitizers should be handled and stored in a manner which prevents their exposure to UV radiation to avoid premature degradation of the photosensitizers.

In some embodiments, the wash composition may contain metal ions, such as iron or copper ions. Any of the multivalent transitional metal ions may also be used. Iron and copper ions enhance the rate of free radical generation from the photosensitizers in the solution. The concentration of these metal ions in the wash composition may be in the range of from 200 ppm to 1000 ppm, or from 300 ppm to 800 ppm, or from 400 ppm to 600 ppm.

In some embodiments, the wash composition may include one or more other enhancers to enhance the efficiency and selectivity of the photosensitizers. Such enhancers include agents to improve the rate of inactivation of microorganisms and are exemplified by adenine, histidine, cysteine, tyrosine, tryptophan, ascorbate, N-acetyl-L-cysteine, propyl gallate, glutathione, mercaptopropionylglycine, dithiothreotol, nicotinamide, butylated hydroxyanisole (BHA), butylated hydroxytoluene (BHT), lysine, serine, methionine, glucose, mannitol, trolox, glycerol, and mixtures thereof.

The wash composition may also include other components such as a buffer, salts, drying agents, antioxidants, and preservatives.

In some embodiments, the wash composition may be an aqueous wash composition. Inclusion of the photosensitizer in the aqueous wash composition may enhance the uniformity of microbial lethality, since the wash composition will typically contact the entire surface of the produce or medical device.

The photosensitizers of the present invention may enhance inactivation of internalized microorganisms since free radicals can penetrate through the intercellular spaces of produce. Also, due to the selection of particular photosensitizers for use in the present invention, significant oxidative stress is only generated upon exposure to UV radiation. Thus, in contrast to conventional oxidants such as hydrogen peroxide and hypochlorite salts, the photosensitizer itself, without application of UV radiation, will not typically cause oxidative stress to the produce.

The photosensitizers of the present invention generate a relatively less offensive flavor and taste profile than conventional oxidants. This is because the photosensitizers of the present invention leave only minimal sensory footprint on the produce.

Embodiments which employ a wash composition as an aqueous wash composition also eliminate a significant drawback associated with fresh produce sanitation, i.e., cross-contamination due to recycling of wash water. The presence of photosensitizers in the wash composition causes formation free radicals upon exposure to UV radiation, which inactivates the microorganisms in the wash composition each time it is used thereby significantly reducing cross-contamination via the wash composition.

The next step in the process of the present invention involves exposure of the associated photosensitizer and surface to UV radiation. As discussed above, the UV radiation induces photolysis of the photosensitizer, which in turn forms free radicals that proceed to inactivate microorganisms associated with the produce and medical device.

The present invention may use UV radiation over the entire ultraviolet spectrum. Wavelengths in the range of from 200 nm to 400 nm, or from 200 nm to 300 nm may also be used. One particularly useful wavelength for the case when fructose forms at least part of the photoinitiator is 254 nm. A skilled person will appreciate that the most suitable wavelength may vary when different photosensitizers are used, because different photosensitizers are most sensitive to UV radiation at different wavelengths.

The time period for exposure to UV radiation should be sufficient to inactivate substantially all of the microorganisms associated with the produce or the medical device. For example, when the photosensitizer is gallic acid or fructose, the exposure time to UV radiation may be from 1 up to 10 minutes, or in the range of from 2 to 8 minutes, or 2 to 5 minutes. In practice, the required time for the UV radiation exposure may be adjusted depending on the level of contamination on the produce, the type of produce being treated, the surface geometry of the produce, concentration of the photosensitizer as well as other factors.

The intensity of the UV radiation, the exposure time and the wavelength are interrelated. For example, the use of a low UV radiation intensity requires a longer exposure time and the use of a higher UV radiation intensity may allow a reduction of the exposure time. In addition, the sensitivity of the photosensitizers to the UV radiation may vary depending on the wavelength of the UV radiation and thus adjustments to the intensity and/or the exposure time may be appropriate depending of the wavelength of UV radiation employed.

Exemplary intensities of UV radiation that can be used may be from 2 mW/cm² to 20 mW/cm², or 3 mW/cm² to 15 mW/cm², or 5 mW/cm² to 10 mW/cm². In one embodiment, the intensity of the UV light is about 8 mW/cm². A variety of instruments are commercially available for measuring UV radiation in the laboratory and in the workplace.

In some embodiments, the UV radiation source and the produce or the medical device may be rotated relative to each other during exposure to the UV radiation. This may be beneficial especially when the photosensitizer-containing composition is sprayed onto the produce or the medical device to provide a thin layer of photosensitizer-containing composition on the surface of the produce or the medical device. Relative rotation can be employed to ensure that more of the surface of the produce or the medical device is exposed to the UV radiation than would be the case without relative rotation. In some embodiments, such as when the produce or the medical device is immersed in a photosensitizer-containing composition, it may be advantageous to stir the photosensitizer-containing composition before and/or during exposure to UV radiation.

The application of UV radiation may be in a variety of forms such as pulsed irradiation or continuous irradiation. In some embodiments, pulsed radiation may be more effective than continuous radiation in creating double strand breaks and irreparable breaks in the DNA or RNA of the microbes. The duration and frequency of pulsing may be adjusting based on the same considerations as discussed above in relation to wavelength, intensity and exposure time.

The UV light source may be a short pulse, high current density, high temperature electric arc having a length of a few mm and being contained within flashbulbs. Such pulsed high-pressure lamps are often xenon flash lamps, which are attractive because a significant fraction of their total light output is in the UV range of the spectrum. This is especially the case for short arc, pulsed xenon lamps that have relatively low output in the red and infrared part of the spectrum and may emit as much as 40% of their total output in the UV range with a wavelength of less than 300 nm.

In one embodiment, the flashbulbs are high pressure, short-arc xenon discharge bulbs, but other discharge gases may be used. Commercial examples of such bulbs typically have an integral reflector that is inside the bulb and a quartz or sapphire window that is highly transmissive of UV radiation. Examples include mercury vapor, mercury vapor with Penning or buffer/diluent mixtures, excimer gases, and other inert gases. A trigger transformer, socket, and related circuit components may be housed in a pulser assembly for each lamp. The flashbulbs may be powered by capacitor discharge. The capacitors may be switched by initiation of the arc in the flashbulbs, which can be triggered by a high voltage trigger pulse. The trigger pulse can be generated by SCR (silicon controlled rectifier) or IGBT (isolated gate bipolar transistor) switching of a trigger capacitor through the pulse transformer of pulser assembly, or other pulsed voltage source.

Another possible UV source adapted for use in the invention comprises one or more linear discharge lamps. These lamps may be continuous discharge lamps or pulsed flash lamps. The output of the UV source unit may be improved by using parabolic reflectors with the discharge lamps placed at the foci of the parabolas. These reflectors may be made from any suitable material as long as the surface adjacent to the lamps is highly UV reflective. Such a reflective surface may comprise a vapor deposited or very highly polished aluminum coating, or a multi-layer dielectric interference coating. The coating may be a vapor deposited aluminum coating on a smooth aluminum substrate, with the aluminum coating also covered by an adhering fused quartz coating or a dielectric coating that protects the reflective nature of the aluminum.

In some embodiments, the UV light source may be connected to a chamber that houses the produce by means of a light guide such as a light channel or fiber optic tube which prevents scattering of the light between the source and the chamber, and more importantly, prevents substantial heating of the produce within the chamber. Direct exposure to the UV light source may raise temperatures as much as 10 to 15° C., especially when the amount of fluid exposed to the UV radiation is small. Use of a light guide may reduce potential heating to less than about 2° C. The method may also include the use of temperature sensors and cooling devices where necessary to keep the temperature of the produce below temperatures at which the produce may be damaged. In some embodiments, the temperature to which the produce is exposed is maintained between about 0° C. to about 10° C., or between about 10° C. and about 45° C., or between about 10° C. and about 37° C., or at about ambient temperature. The process of the present invention may be carried out in batch-wise or continuous fashion.

The present invention may also be used for waste water treatment. The photosensitizers of the present invention may be added to the waste water, which is then exposed to UV light. The free radicals thus generated induce oxidative stress, which oxidize waste materials such as organic matter and hazardous chemicals in the waste water. Furthermore, the microorganisms in the waste water will also be inactivated.

The present invention has several advantages for use in sanitation of produce/medical devices, namely, (1) effective and uniform microbial inactivation, (2) ability to inactivate both bacteria and viruses on the surface of produce/medical device, even inside the produce matrix, (3) use of photosensitizers that are safe from both the environmental and health perspectives, and (4) use of photosensitizers that are generally regarded as safe from a ‘clean label’ perspective. The present invention can provide a safe and cost effective method for improved sanitation of produce to extend shelf life and with little impact on product quality.

### EXAMPLES

### Example 1

In this example, fluorescein dye was used to detect the presence of free radicals, since interactions of oxidizing free radicals with fluorescein quench the fluorescence signal intensity. A fluorescein dye solution (4 μg/L) containing 0.4% (w/v) fructose and a fluorescein dye solution (4 μg/L) without fructose (control) were exposed to UV radiation at wavelength of 254 nm. Referring to FIG. 2, when fructose was present in the solution, the fluorescence intensity of the exposed fluorescein dye rapidly decreased, in contrast to the relatively constant fluorescence intensity of the exposed fluoroscein dye in the absence of fructose. These results provide a clear indication that exposure of fructose to UV radiation at a wavelength of 254 nm resulted in generation of a significant quantity oxidizing free radicals, as evidenced by the significant reduction in fluorescence resulting from oxidation of the fluoroscein dye by the oxidizing free radicals.

### Example 2

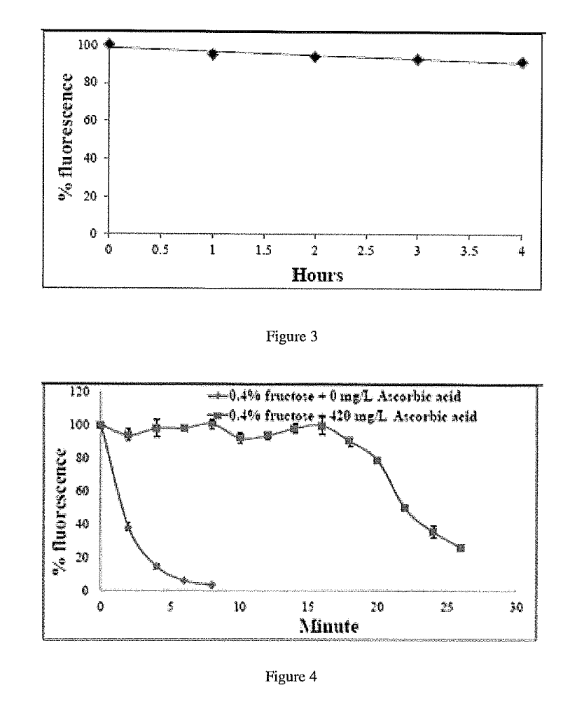

In this example, a study was conducted to evaluate if the free radicals were generated only upon exposure to UV radiation. The fluorescence intensity of the fluorescein dye in 0.4% fructose solution after 4 minutes of UV radiation exposure, i.e. in a post-UV processing storage phase, was measured. The measurement showed no significant changes in fluorescence intensity in the absence of UV radiation (FIG. 3). These results indicate that a substantial amount of free radicals are generated by the fructose photosensitizer only in presence of UV radiation.

### Example 3

In this example, ascorbic acid, an antioxidant compound, was used to counter the oxidative effect of the generated free radicals on the fluorescein dye. A fluorescein dye solution (4 μg/L) containing 0.4% (w/v) fructose and 420 mg/L of ascorbic acid was compared to a fluorescein dye solution (4 μg/L) containing only 0.4% (w/v) fructose. During exposure of the solutions to UV radiation at a wavelength of 254 nm, the fluorescence intensities of the solution were measured. Referring to FIG. 4, the rate of loss of fluorescence was significantly reduced due to the antioxidant activity of ascorbic acid, since the ascorbic acid is preferentially oxidized by free radicals generated by exposure of the fructose to UV radiation. As a result, the rate of change in the fluorescence was decreased since less free radicals were available to react with the fluoroscein dye.

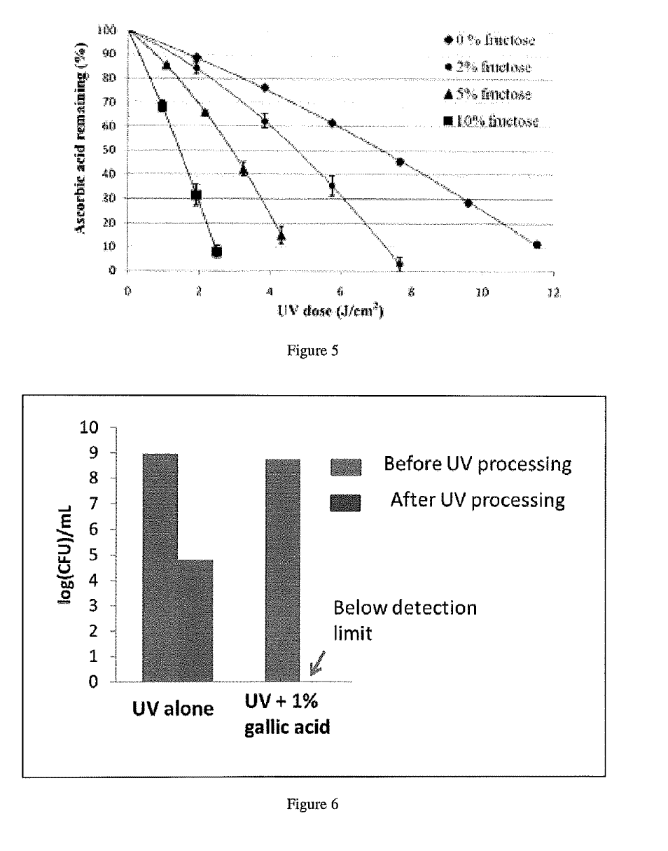

UV induced degradation of ascorbic acid was also measured in this example. Different concentrations of fructose were used in solutions containing 100 mg/L of ascorbic acid. Upon exposure to different doses of UV radiation at a wavelength of 254 nm, the remaining amount of ascorbic acid was determined (FIG. 5). The results demonstrate that the rate of ascorbic acid degradation increased as the concentration of fructose was increased. These results indicate a direct correlation between the fructose concentration and the amount of free radical generation. Also, these results show that higher doses of UV radiation lead to a greater amount of free radical generation which manifests itself as lower ascorbic acid contents in the solutions.

### Example 4

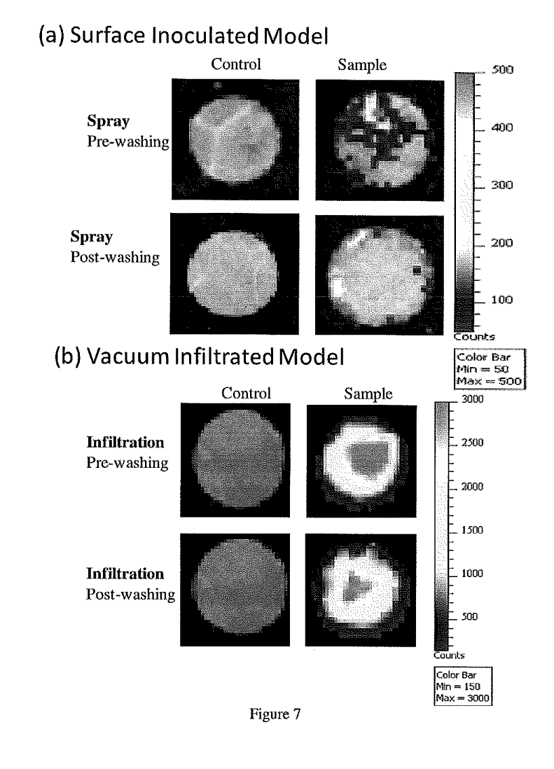

E. coli BL-21 was suspended in phosphate buffer at the level of 109 CFU/mL. Gallic acid was incorporated into bacterial suspension at the level of 1% (w/v). This suspension was exposed to UV light (intensity of 4 mJ/cm²) for 10 seconds. The control experiment was performed in the exact same manner except for addition of gallic acid. After UV treatment, the cells were separated from the buffer through centrifugation and bacterial inactivation was measured using a plate count technique. The results are presented in FIG. 6.

After exposure to UV light, the microbial count was reduced to undetectable when 1% gallic acid was used. While for the control group where on UV light was used with no photosensitizer, significant microbial count remains in the system (FIG. 6).

### Example 5

Fructose, sucrose, glucose, the sodium salt of fluorescein, furan, ascorbic acid, and 30% (w/w) hydrogen peroxide were obtained from Sigma Aldrich (St. Louis, Mo.). A batch-UV processing unit (Spectronics Spectrolinker XL-1500 UV Crosslinker, Westbury, N.Y.) was used for Examples 5-10. The apparatus consisted of 5 UV lamps (254 nm, 15 W, Spectronics Corporation, Westbury, N.Y.) that generated a UV intensity of approximately 20 mW/cm² at the surface of exposure mounted within a shielded box (46.4×15.9×31.8 cm). Fluorescence intensity measurement noise was minimized by allowing the lamps to warm up for at least 15 minutes prior to taking measurements.

The test fluorescein solution with approximately 1 μM fluorescein was prepared in deionized water (pH 6.3) or 100 mM buffer at pH 6. The effect of various compounds (fructose, sucrose, glucose, sodium salt of fluorescein, furan, and ascorbic acid) on the decay rate of fluorescence from fluorescein was investigated by dissolving these compounds individually in the fluorescein solution and exposing the solution to UV light (Examples 6-10). Specifically, treatments were carried out by adding a 10.0 ml solution into an uncovered glass petri dish and exposing it to UV radiation for various amounts of time (0-12 minutes) in the UV processing unit. The samples in the petri dish were stirred to achieve uniform exposure to UV light. Ambient room temperature (20-22° C.) was used for the treatments. To measure the fluorescence intensity of the solution, at each time interval, 100 μl of the sample was pipetted from the petri dish into a well of a 96-well plate optimized for fluorescence measurement. Fluorescence was measured in a Gemini XPS fluorescence micro-plate reader (Molecular Devices, Sunnyvale, Calif.) with excitation and emission wavelengths of 485 nm and 510 nm, respectively. All the fluorescence values were normalized using Eq. (1):

$$Relative fluorescence intensity = \frac{100 \times {I}_{t}}{{I}_{0}} \qquad ( 1 )$$

where I₀=fluorescence intensity at time t=0 minutes and Iₜ=fluorescence intensity after ‘t’ minutes of UV exposure.

### Example 6

To examine the effect of sugars such as fructose, glucose and sucrose on the fluorescence decay rate of fluorescein upon exposure to UV light, each of the sugars was separately dissolved in 1 μM fluorescein solution at the level of 263 mM for sucrose (9% w/v) and 500 mM for glucose and fructose (9% w/v). These solutions were subsequently exposed to UV light for up to 12 minutes.

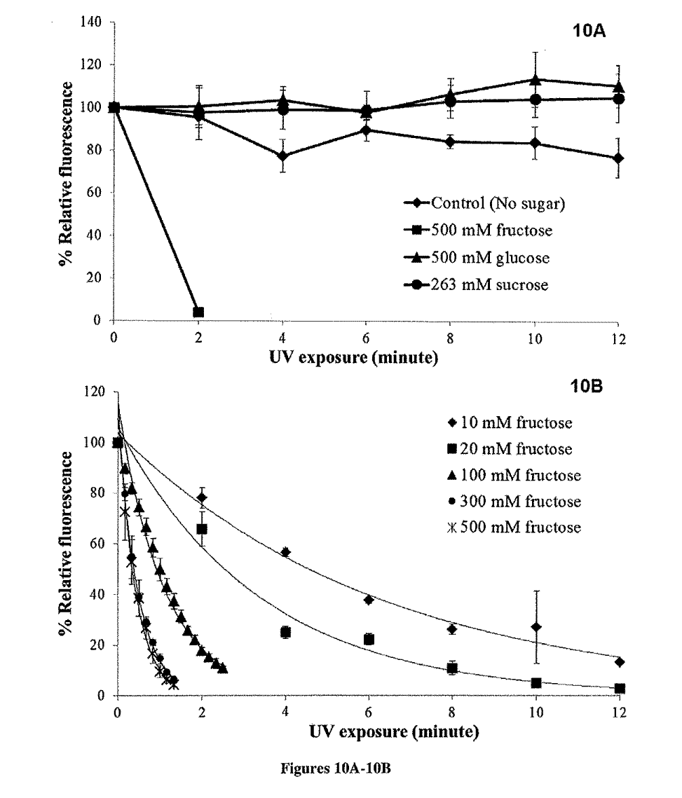

FIG. 10A shows the decay of fluorescence intensity from fluorescein as a function of the duration of exposure to UV light in the presence of 263 mM sucrose, 500 mM glucose and 500 mM fructose. In the absence of sugar (negative control), the fluorescein solution showed an approximately 20% decrease in fluorescence, possibly due to trace amounts of oxidative stress generated within the solution. Sucrose and glucose had no effect on the fluorescence intensity of fluorescein, indicating that sucrose and glucose did not generate oxidizing species during 12 minutes of UV exposure. However, the presence of fructose in the fluorescein solution caused a significant decrease in the fluorescence intensity values and more than 90% of fluorescence was lost within 2 minutes of exposure to UV light (FIG. 10A). The average absorbance values for 500 mM glucose and 263 mM sucrose solutions at 254 nm were less than 0.001, while the solution containing 500 mM fructose showed an absorbance value of 0.12. Thus, differences in solution absorbance did not cause the dramatic decrease in the fluorescence intensity observed in the presence of fructose. It was observed separately that mere addition of fructose to the fluorescein solution in the absence of UV light did not show any effect on the fluorescence intensity values of fluorescein.

To further validate the effect of fructose, various fructose concentrations (10, 20, 100, 300 and 500 mM) were dissolved in 1 μM fluorescein solution and exposed to UV light. Fluorescence decay caused by fructose followed first order kinetics (r²>0.9) at all concentrations of fructose used in this example (FIG. 10B). The decay rate constant values in the presence of 10, 20, 100, 300 and 500 mM fructose were 0.16±0.01, 0.27±0.02, 0.91±0.02, 2.1±0.06 and 2.4±0.13 min⁻¹, respectively. Statistical analysis of the rate constant values suggested that the degradation rate increased with fructose concentration up to 300 mM (F-test, p<0.05). However, there was no significant difference between the fluorescence decay rates for 300 and 500 mM fructose concentrations, suggesting that at these concentrations, fructose was present in excess. The lowest concentration of fructose used in this example (10 mM) was approximately 10,000-fold higher than the fluorescein concentration (about 1 μM). Thus, in comparison to fluorescein concentration, a large amount of fructose was needed to accomplish the oxidation of fluorescein.

### Example 7

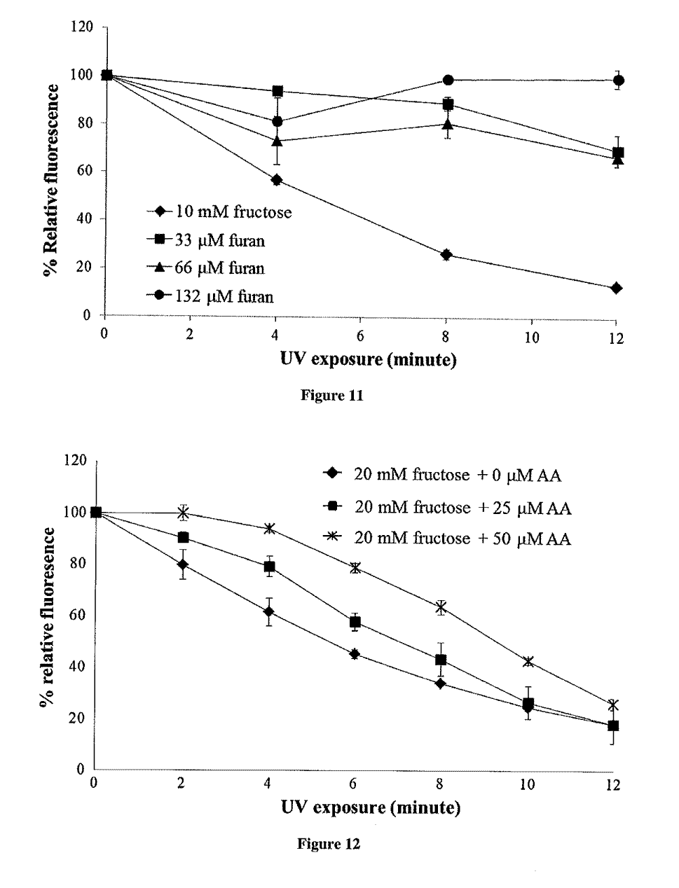

The effect of furan on fluoresce decay was tested in this example. Furan was added to a fluorescein solution at 33, 66 and 132 μM levels prior to UV exposure. The fluorescence decay rate of fluorescein in the presence of various concentrations of furan is shown in FIG. 11. At levels of 33 and 66 μM, furan had only a marginal effect (<20% change) on the relative fluorescence intensity after 12 minutes of UV exposure, while furan at 132 μM showed no effect. These results show that the presence of furan did not cause oxidation of fluorescein. At the highest concentration of 132 μM, the average absorbance of furan at 254 nm was less than 0.001. This study suggests that the fluorescence quenching effect of UV exposed fructose was not from the stable furan product formed as a result of UV exposure of fructose, but instead due to transient intermediates formed during UV-induced fructose degradation.

### Example 8

This study tested the effect of added antioxidant on the rate of fluorescence decay. Ascorbic acid (AA) was added at concentrations of 25 and 50 μM to a 1 μM fluorescein solution containing 20 mM fructose prepared in 100 mM phosphate buffer (pH 6). The solutions were prepared in phosphate buffer to minimize pH change after addition of ascorbic acid. The fluorescence decay rate of fluorescein in these solutions is shown in FIG. 12, which shows the rate of loss of fluorescence of fluorescein in the presence of 20 mM fructose and various concentrations (0-50 μM) of ascorbic acid. The rate of fluorescence decay was significantly reduced after addition of ascorbic acid and the effect was concentration dependent (p<0.05). Since ascorbic acid is a known antioxidant with the ability to quench free radicals, these results demonstrate that the photolysis products of fructose are oxidative.

### Example 9

The effect of dissolved oxygen on the generation of oxidative species from photolysis of fructose was tested in this example. Experiments were performed in the presence or absence of atmospheric oxygen. Quartz cuvettes were filled with 1 μM fluorescein solution containing 500 mM fructose and exposed to nitrogen for 5 minutes and immediately sealed. These sealed quartz cuvettes were subsequently exposed to UV light for 60 seconds and the fluorescence of the solution was measured. The control for this test consisted of a 1 μM fluorescein solution containing 20 mM fructose filled in quartz cuvettes and exposed to UV light without prior nitrogen purging.

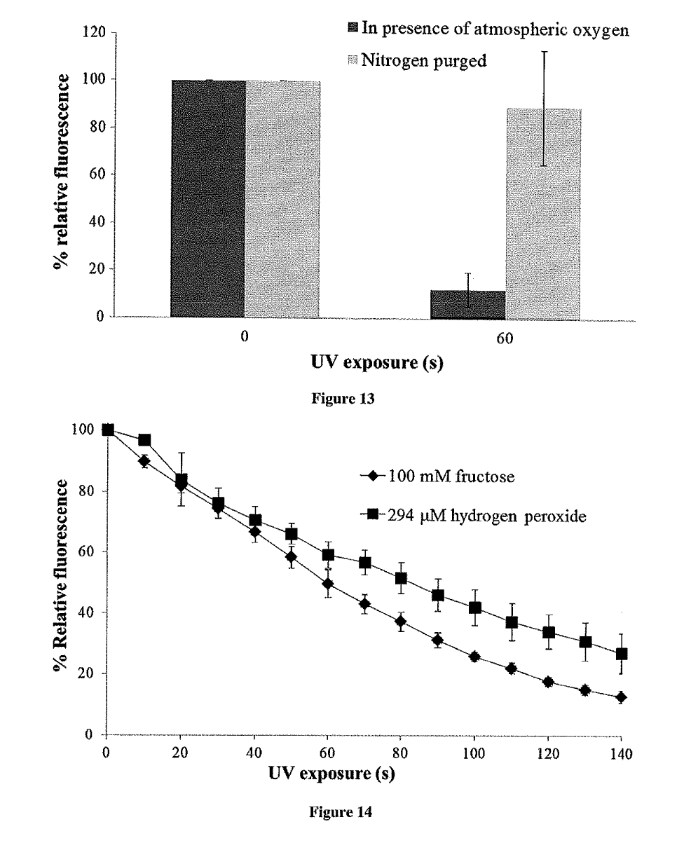

FIG. 13 shows the rate of fluorescence decay of fluorescein in 500 mM fructose solutions exposed to UV light with or without nitrogen purging. After 1 minute of exposure to UV light, approximately 90% of the fluorescence remained in samples purged with nitrogen, while only 11% of fluorescence remained in samples not purged with nitrogen. The results demonstrate that oxygen plays a significant role in either generation or propagation of reactive oxygen species generated upon UV exposure of fructose.

### Example 10

The oxidative effect of fructose on fluorescence decay was quantitatively compared with that of hydrogen peroxide, a compound known to produce oxidative species upon exposure to UV light. Hydrogen peroxide was added to a 1 μM fluorescein solution to a final concentration of 294 μM (0.001% w/v). This solution was subsequently exposed to UV light and the fluorescence of the sample was measured at 10 second intervals. Fructose was added at a level of 100 mM in a 1 μM fluorescein solution and the experiment was performed in a similar manner. The % relative fluorescence was plotted against the duration of UV exposure (FIG. 14). The area under the curve for each sample was calculated using the formula for the area of trapezium as shown in Eq. (2):

$$AUC = ( Δ t ) \frac{f ( t ) + f ( t + Δ t )}{2} \qquad ( 2 )$$

where t is the time in minutes and f is the relative fluorescence intensity.

Relative oxidative potential was calculated by comparing the AUC values for an individual compound (fructose and hydrogen peroxide) and the respective molarities of these compounds in the solutions as shown in Eq. (3):

$$Relative oxidative potential = \frac{{AUC}_{Fructose} \times {M}_{Hydrogen peroxide}}{{AUC}_{Hydrogen peroxide} \times {M}_{Fructose}} \qquad ( 3 )$$

where, M is the molarity of either fructose or hydrogen peroxide in the solutions.

FIG. 14 shows the rate of fluorescence decay of fluorescein incubated with either a 294 μM hydrogen peroxide (0.001% w/v) solution or a 100 mM fructose solution when exposed to UV light. Quantitative comparison between the two compounds was performed by comparing the areas under the curves and their respective molarities. Based on these calculations, the relative oxidation potential of UV exposed fructose was approximately 0.0025 compared to hydrogen peroxide. This shows that only a small fraction of fructose (0.8%) was in a form that exhibited photosensitivity to UV light. However, fructose can occur in fruit products at the levels used in this study (up to 9% w/v) and, as a result, the oxidative effect of fructose can be comparable to that of hydrogen peroxide. The results of this study highlight the oxidative nature of UV exposed fructose, because the majority of fruit and juices contain fructose.

### Comparative Example A

In this example, a study was conducted to determine the effect simple washing on microorganism content. The study used a combination of bioluminescence and traditional plate counting methods to enumerate microorganisms on fresh lettuce leaf samples. LuxCDABE-expressing E. coli bacterial cells on intact lettuce leaf samples were contacted with a simple washing solution and the samples were imaged using bioluminescence imaging. The results of wide-field bioluminescence imaging of the bacteria on intact leaf samples are presented in FIG. 7. These wide-field bioluminescence imaging results show an overlay of bioluminescence signal intensity over a white light image of a lettuce leaf. From these images, it is clear that wide-field bioluminescence imaging is an appropriate method for enumerating microorganisms.

This comparative example also compared the efficiency of a simple washing procedure to remove surface inoculated and internalized bacteria (vacuum infiltrated bacteria). The surface inoculated bacterial cells were easily removed (more than 90% of cells were removed as shown in FIG. 7(a)) while only a limited number of infiltrated bacterial cells (less than 10% of cells as shown in FIG. 7(b)) could be removed from lettuce samples with only simple washing. In the case of the surface inoculated model, the imaging data showed retention of a small number of bacterial cells only along the cut edge of the lettuce disk, while in the case of the infiltrated bacterial cells a large number of bacterial cells were retained in the center of the leaf sample even after washing.

### Comparative Example B

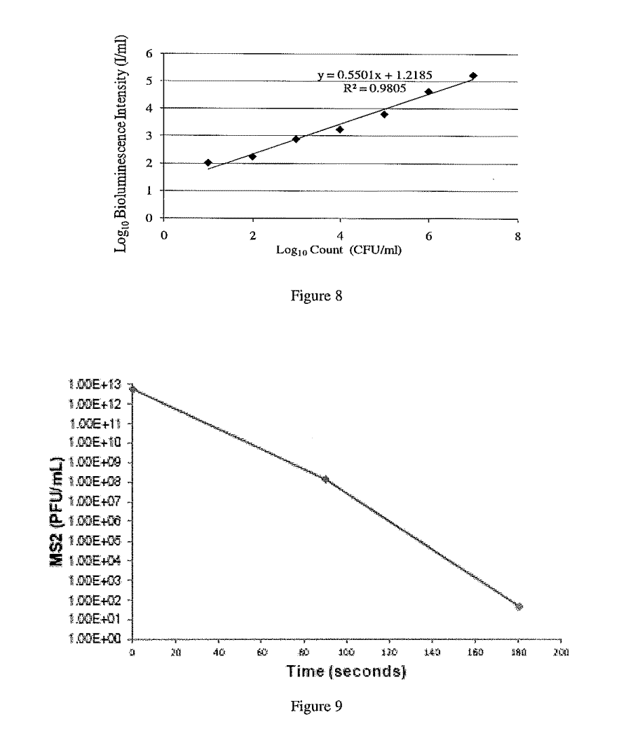

In this example, a study was conducted to determine the sensitivity of wide-field bioluminescence imaging and its correlation with plate counts of bacterial cells. In this example, the bacterial cells on leafy greens were treated with T4 phages. The results shown in FIG. 8 demonstrate the high sensitivity and quantitative ability of bioluminescence imaging. The minimum detectable concentration of E. coli for wide-field bioluminescence imaging was approximately 100 CFU/5 cm² of lettuce sample (FIG. 8). The sensitivity of wide-field imaging is limited by the ability of ICCD camera, background noise in the imaging system and the limited amount of auto luminescence of plant leafs. The signal intensity for 100 CFU/5 cm² was three times higher than the background bioluminescence intensity of leaf tissue. These results also show a linear relationship between the bioluminescence signal intensity and the bacterial concentration and agree with predicted values.

### Comparative Example C

In this example, a study was conducted to quantify the inactivation of viral particles upon exposure to UV radiation in water. In this example, the bacterial cells on leafy greens were exposed to UV radiation in water without a photosensitizer. FIG. 9 shows rapid and effective inactivation of the MS2 viral particles (approximately an 11 log reduction by exposure to UV radiation for three minutes.

It is to be understood, however, that even though numerous characteristics and advantages of the present invention have been set forth in the foregoing description, together with details of the structure and function of the invention, the disclosure is illustrative only, and changes may be made in detail, especially in matters of shape, size and arrangement of parts within the principles of the invention to the full extent indicated by the broad general meanings of the terms in which the appended claims are expressed.

What is claimed is:

1. A process for treatment of a surface comprising the steps of:
associating a surface with at least one photosensitizer selected from the group consisting of gallic acid, riboflavin, photo-porphyrin, sodium chlorophyllin and fructose; and
exposing the associated photosensitizer and the surface to UV radiation to cause the photosensitizer to generate one or more free radicals;
wherein the surface is a surface of a produce or a surface of a medical device.

2. The process of claim 1, wherein the produce is selected from fresh vegetables, fruits and cut fruits.

3. The process of claim 1, wherein the associating step comprises a contacting step wherein a wash composition containing the photosensitizer contacts the surface.

4. The process of claim 3, wherein the contacting step is selected from the group consisting of immersing the produce in the wash composition, spraying the produce with the wash composition, dipping the produce into the wash composition, and wiping the produce with the wash composition.

5. The process of claim 4, wherein in said contacting step the produce is immersed in the wash composition.

6. The process of claim 4, wherein in said contacting step the produce is sprayed with the wash composition.

7. The process of claim 3, wherein the wash composition is an aqueous solution.

8. The process of claim 7, wherein the aqueous solution has a pH in the range of from about 3 to about 7.

9. The process of claim 8, wherein the aqueous solution has a photosensitizer concentration in the range of from 0.1 w/v % to 10 w/v %.

10. The process of claim 8, wherein the wash composition has a photosensitizer concentration in the range of from 0.2 w/v % to 5 w/v %.

11. The process of claim 8, wherein the wash composition has a photosensitizer concentration in the range of from 0.5 w/v % to 3 w/v %.

12. The process of claim 1, wherein the one or more photosensitizers is associated with the produce at a temperature in the range of from 10° C. to 50° C.

13. The process of claim 1, the one or more photosensitizers is associated with the produce at a temperature in the range of from 20° C. to 35° C.

14. The process of claim 1, wherein the one or more photosensitizers are in a wash composition for the produce when associated with the produce.

15. The process of claim 1, wherein the UV radiation has a wavelength in the range of from 200 nm to 400 nm.

16. The process of claim 15, wherein the exposing step is conducted over a period of from 2 to 8 minutes.

17. The process of claim 1, wherein the UV radiation has an intensity of from 2 mW/cm² to 20 mW/cm².

18. The process of claim 1, wherein the produce is contaminated with microorganisms selected from bacteria, fungi, and viruses, or pesticides.

19. The process of claim 1, wherein the medical device is contaminated with microorganisms selected from bacteria, fungi, and viruses.

20. A process of treatment of waste water, comprising the steps of:
adding at least one photo sensitizer selected from the group consisting of gallic acid, riboflavin, photo-porphyrin, sodium chlorophyllin and fructose to waste water; and
exposing the waste water with the photosensitizer to UV radiation to cause the photosensitizer to generate one or more free radicals.
